(19) United States
(12) Patent Application Publication
(10) Pub. No.: US 2006/0247657 A1
(43) Pub. Date: Nov. 2, 2006

(54) Methods and systems for characterizing intervertebral disc space

(75) Inventor: Hai H. Trieu, Cordova, TN (US)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(73) Assignee: SDGI HOLDINGS, INC.

(21) Appl. No.: 11/115,229

(22) Filed: Apr. 27, 2005

(51) Int. Cl. A61B 5/107

(52) U.S. Cl. 606/102; 600/587

(57) ABSTRACT

A system and method for determining at least one parameter of an intervertebral space. The system and method may be useful to determine the appropriate size and geometry of a spinal implant. An expandable member may be inserted into an intervertebral disc space and inflated with an imaging contrast medium. The inflated expandable member and at least the intervertebral disc space may be imaged. The volume of imaging contrast medium in the expandable member may be measured in order to determine the volume of the disc space.

### FIELD OF THE INVENTION

Embodiments relate to methods and systems for characterizing the intervertebral disc space. More particularly, embodiments of the invention relate to methods and systems for measuring the volume and other parameters, as well as determining the geometry of the intervertebral disc space using expandable members and imaging contrast media.

### BACKGROUND OF THE INVENTION

The intervertebral disc functions to stabilize the spine and to distribute forces between vertebral bodies. The intervertebral disc usually includes three structures: the nucleus pulposus, the annulus fibrosis, and two vertebral end-plates. The nucleus pulposus is an amorphous hydrogel in the center of the intervertebral disc. The annulus fibrosis, which is comprised mostly of highly structured collagen fibers, maintains the nucleus pulposus within the center of the intervertebral disc. The vertebral end-plates, primarily comprised of hyalin cartilage, separate the disc from adjacent vertebral bodies and act as a transition zone between the hard vertebral bodies and the soft disc.

Intervertebral discs may be displaced or damaged due to trauma, disease, or the normal aging process. One way to treat a displaced or damaged intervertebral disc is by surgical removal of a portion or all of the intervertebral disc, including the nucleus and the annulus fibrosis. However, the removal of the damaged or unhealthy disc may allow the disc space to collapse, which may lead to instability of the spine, abnormal joint mechanics, nerve damage, and severe pain. Therefore, after removal of the disc, a spinal implant such as a prosthetic nucleus, artificial disc, or fusion cage may be implanted in order to replace the removed nucleus or annulus, or a portion thereof.

Because the spinal implant is replacing all or part of the intervertebral disc, it may be desirable to size the spinal implant according to the natural dimensions and geometry of the intervertebral disc that is to be replaced or augmented.

The description herein of problems and disadvantages of known devices and methods is not intended to limit the invention to the exclusion of these known entities. Indeed, embodiments of the invention may include one or more of the known devices and methods without suffering from the disadvantages and problems noted herein.

### SUMMARY OF THE INVENTION

What is needed are systems and methods for determining various parameters of the intervertebral disc space such as the volume, dimensions, and geometry. Embodiments of the invention solve some or all of these needs, as well as additional needs.

Therefore, in accordance with an embodiment of the present invention, there is provided a method for determining at least one parameter of an intervertebral disc space. An expandable member may be inserted into the intervertebral disc space. The expandable member may be inflated with an imaging contrast medium, deflated, and removed from the disc space.

In another embodiment, there is provided another method for determining at least one parameter of an intervertebral disc space. At least a portion of a nucleus and/or annulus of the intervertebral disc may be removed. An expandable member may be inserted into the disc space and inflated with an imaging contrast medium. The volume of the imaging contrast medium used to inflate the expandable member may be measured. Additionally, the expandable member may be imaged while inflated with the imaging contrast medium. The expandable member may be deflated and removed from the disc space.

In another embodiment, there is provided a device for determining at least one parameter, such as the volume, dimensions, and geometry, of an intervertebral disc space. The device may comprise a longitudinal element. An expandable member comprising an internal cavity may be connected to and in communication with the distal end of the longitudinal element. A detachable syringe may be connected to the proximate end of the longitudinal element. The device also may comprise an imaging contrast medium.

In another embodiment, there is provided a surgical kit comprising a longitudinal element having distal and proximate ends; an expandable member; a syringe; and an imaging contrast medium.

These and other features and advantages of the present invention will be apparent from the description provide herein.

### BRIEF DESCRIPTION OF THE DRAWINGS

FIG. 1, embodiments A and B, is a drawing of an exemplary device according to embodiments of the invention.

FIG. 2, embodiments A and B, is a drawing of another exemplary device according to embodiments of the invention.

FIG. 3 is a drawing of another exemplary device according to embodiments of the invention.

FIG. 4 embodiments A, B, and C, is a drawing of an exemplary method according to embodiments of the invention.

FIG. 5 is a drawing of another exemplary device according to embodiments of the invention.

FIG. 6, embodiments A and B, are in-vivo images of an exemplary device according to embodiments of the invention.

### DETAILED DESCRIPTION OF THE EMBODIMENTS

The following description is intended to convey a thorough understanding of the various embodiments of the invention by providing a number of specific embodiments and details involving systems and methods for determining at least one parameter of the intervertebral disc space, in particular for measuring the volume, dimensions, and geometry of the intervertebral disc space. It is understood, however, that the present invention is not limited to these specific embodiments and details, which are exemplary only. It is further understood that one possessing ordinary skill in the art, in light of known systems and methods, would appreciate the use of the invention for its intended purposes and benefits in any number of alternative embodiments.

Throughout this description, the expression “intervertebral disc space” may refer to any volume between two adjacent vertebrae. The intervertebral disc space may be the volume inside of the annulus fibrosis of the intervertebral disc. Alternatively, the intervertebral disc space also may include the annulus fibrosis itself.

It is a feature of an embodiment of the present invention to provide a device for determining at least one parameter, such as the volume, dimensions, and geometry, of an intervertebral space. The device may comprise a longitudinal element comprising distal and proximate ends. An expandable member comprising an internal cavity may be connected to and in communication with the distal end of the longitudinal element. A detachable syringe optionally may be connected to the proximate end of the longitudinal element. An imaging contrast medium may be used to inflate the expandable member.

The longitudinal element may be used to deliver the imaging contrast medium to the internal cavity of the expandable member. The longitudinal element may have an optimal stiffness and flexibility to facilitate insertion into the body and maneuverability. In a preferred embodiment, the distal end of the longitudinal element may be curved or easily deformable to conform to the intervertebral disc space. Additionally, the longitudinal element may have an optimal diameter for insertion into the body and delivery of the expandable member to the intervertebral disc space. It may be preferable that the diameter of the longitudinal element be not more than the height of the disc space, for example no more than about 12 mm, preferably no more than about 10 mm, and most preferably no more than about 8 mm in diameter. This may allow the longitudinal element to be inserted into the intervertebral disc space for delivery of the expandable member therein. One who is skilled in the art will appreciate how to choose the appropriate size and flexibility of the longitudinal element in accordance with the limitations described herein.

The expandable member may be connected to and in communication with the distal end of the longitudinal element. The expandable member may be an appropriate, biocompatible member having an internal cavity. Because the expandable member preferably is inserted into the body only for a momentary period of time, the expandable member need not be as biocompatible as a permanent implant. However, it is preferable that the expandable member be sufficiently biocompatible as to not cause any undesirable interactions during its brief insertion into the body.

The expandable member preferably may be selected to withstand the pressure of inflation when the imaging contrast medium is delivered to the expandable member so as to avoid rupture when inflated. Rupture could cause a leak of potentially toxic or otherwise dangerous imaging contrast medium into the body and preferably may be avoided.

In a preferred embodiment, the expandable member is a balloon. The balloon may be made of various polymeric materials such as polyethylene terephthalates, polyolefins, polyurethanes, nylon, polyvinyl chloride, silicone, polyetherketone, polylactide, polyglycolide, poly(lactide-co-glycolide), poly(dioxanone), poly([epsilon]-caprolactone), poly(hydroxylbutyrate), poly(hydroxylvalerate), tyrosine-based polycarbonate, polypropylene fumarate, and mixtures and combinations thereof. Because the balloon is intended to be filled with image contrast agents and/or radioactive materials, it is preferred to fabricate the balloon from chemical resistant materials. In addition, balloon may be made from a multi-layered material with an inner expandable chemically resistant layer, and/or the interior of the balloon may be coated with a chemically resistant coating.

In a more preferred embodiment, the expandable member is an unconstrained balloon. An unconstrained balloon is generally spherical or cylindrically shaped, and expands roughly equally in all directions when inflated so long as it is not constrained by surrounding tissues (e.g. the interior surfaces of an intervertebral disc space). By comparison, the expansion of a constrained balloon may be limited by its inherent properties and/or materials in order to expand preferentially or selectively in certain directions when inflated. A constrained balloon, for example, may be shaped like a kidney or flattened disk when inflated. The preferred unconstrained balloon of the present invention may be desirable because it can conform to volumes of varying geometry, whereas the constrained balloon is best suited to conforming to volumes shaped similar to the shape of the constrained balloon. Therefore, an unconstrained balloon may be more adaptable to various intervertebral disc space volumes than is a constrained balloon

A detachable syringe may be connected to the proximate end of the longitudinal element. The detachable syringe may be detachably connected to the longitudinal element using any appropriate detachment means. In a preferred embodiment, the detachable syringe may be connected to the proximate end of the longitudinal element using a luer lock. Alternatively, the proximate end of the longitudinal element may include a seal that can be repeatedly punctured by a needle, much like a medicine vial. Other connection devices including, but not limited to, luer slip connectors, also may be used to detachably connect the syringe to the proximate end of the longitudinal element. The detachable syringe may be used to draw an imaging contrast medium from a separate container and then deliver the imaging contrast medium to the longitudinal element. In a preferred embodiment, the detachable syringe may be graduated by volume so that the volume of imaging contrast medium delivered may be easily measured.

The imaging contrast media contemplated for use in the embodiments include all applicable imaging contrast media, including contrast agents for X-ray, derivative X-ray technologies such as CT (computerized tomography) and C-arm fluoroscopy (e.g. Iso-C technology available from Siemens AG, Berlin, Germany), MRI (magnetic resonance imaging), and PET (positron emission tomography) imaging. Typically, the imaging contrast medium may be chosen to correspond to the imaging technique to be used. For example, if X-ray images are to be taken of the inflated expandable member, then X-ray imaging contrast media preferably may be used. Similarly, if images are to obtained using an MRI technique, then MRI imaging contrast media preferably may be used. Additionally, it may be preferable that the imaging contrast medium comprise a fluid or liquid solution, gel, paste, or suspension of an X-ray, CT, MRI, and PET contrast agent rather than an aqueous composition containing the contrast agent. Therefore, it should be understood that imaging contrast media may comprise fluid or liquid solutions, gels, pastes, and suspensions of X-ray, CT, MRI, and PET contrast agents in addition to the contrast agent itself. One who is skilled in the art will appreciate the wide array of imaging contrast media that may be used in accordance with the invention.

Specific X-ray imaging contrast media contemplated for use in the embodiments include, but are not limited to, barium sulfate, acetrizoic acid derivatives, diatrizoic acid derivatives such as Hypaque® (commercially available from Amersham, GE Healthcare, Chalfont St. Giles, United Kingdom), diatrizoate meglumine/sodium, iothalamic acid derivatives, iothalamates, ioxithalamic acid derivatives, iothalamate meglumine, metrizoic acid derivatives, iodamide, iodipamide meglumine, ioglycamic acid, dimeric ionic contrast agents, ioxaglic acid derivatives, metrizamide, metrizoate, iopamidol, iohexol, iopromide, iobitridol, iomeprol, iopentol, ioversol, ioxilan, iodixanol, iotrolan, ioxaglate (Hexabrix®, commercially available from Mallinckrodt Imaging, Tyco Healthcare, Mansfield, Mass.), ioxaglate meglumine/sodium, iotrol, iopanoic acid, and organic radiographic iodinated contrast media (ICM) such as modifications of a 2,4,6-tri-iodinated benzene ring including Renografin® (commercially available from Amersham, GE Healthcare, Chalfont St. Giles, United Kingdom), Conray® (commercially available from Mallinckrodt Imaging, Tyco Healthcare, Mansfield, Mass.), iohexol (Omnipaque®, commercially available from GE Healthcare, Chalfont. St. Giles, United Kingdom), iopamidol (Isovue®, commercially available from Bracco Diagnostics, Princeton, N.J.), ioversol (Optiray®, commercially available from Mallinckrodt Imaging, Tyco Healthcare, Mansfield, Mass.), and iopromide (Ultravist®, commercially available from Berlex Imaging, Montville, N.J.).

Specific MRI imaging contrast media contemplated for use in the embodiments include, but are not limited to, gadolinium derivatives and complexes such as gadoteridol, gadoterate meglumine, gadodiamide, and gadopentetate (Magnevis®, commercially available from Berlex Imaging, Montville, N.J.); iron derivatives and complexes; manganese derivatives and complexes such as mangafodipir trisodium; superparamagnetic iron oxide contrast medias; ferumoxides such as FERIDEX® (commercially available from Berlex Imaging, Montville, N.J.); and perfluorocarbons. The MRI imaging contrast media may be either positive or negative contrast media.

It may be desirable that the MRI imaging contrast media comprise complexes of a complexing agent and a metal such as gadolinium, manganese, or iron. Exemplary complexing agents include, but are not limited to, diethylenetriamine-pentaacetic acid (“DTPA”); 1,4,7,10-tetraazacyclododecane-N,N′,N″,N′″-tetraacetic acid (“DOTA”); p-isothiocyanatobenzyl-1,4,7,10-tetraazacyclododecane-1,4,7,10-tetraacetic acid (“p-SCN-Bz-DOTA”); 1,4,7,10-tetraazacyclododecane-N,N′,N″-triacetic acid (“DO3A”); 1,4,7,10-tetraazacyclododecane-1,4,7,10-tetrakis(2-propionic acid) (“DOTMA”); 3,6,9-triaza-12-oxa-3,6,9-tricarboxymethylene-10-carboxy-13-phenyl-tridecanoic acid (“B-19036”); 1,4,7-triazacyclononane-N,N′,N″-triacetic acid (“NOTA”); 1,4,8,11-tetraazacyclotetradecane-N,N′,N″,N′″-tetraacetic acid (“TETA”); triethylene tetraamine hexaacetic acid (“TTHA”); trans-1,2-diaminohexane tetraacetic acid (“CYDTA”); 1,4,7,10-tetraazacyclododecane-1-(2-hydroxypropyl)4,7,10-triacetic acid (“HP-DO3A”); trans-cyclohexane-diamine tetraacetic acid (“CDTA”); trans(1,2)-cyclohexane diethylene triamine pentaacetic acid (“CDTPA”); 1-oxa-4,7,10-triazacyclododecane-N,N′,N″-triacetic acid (“OTTA”); 1,4,7,10-tetraazacyclododecane-1,4,7,10-tetrakis {3-(4-carboxyl)-butanoic acid}; 1,4,7,10-tetraazacyclododecane-1,4,7,10-tetrakis(acetic acid-methyl amide); 1,4,7,10-tetraazacyclododecane-1,4,7,10-tetrakis(methylene phosphonic acid); and derivatives and analogs thereof, particularly protected forms of the compounds.

CT scan imaging contrast media contemplated for use in the embodiments include orally, intravenously, and rectally administered media. Specific CT scan imaging contrast media contemplated for use in the invention include, but are not limited to, iodine solutions, barium sulfate, mixtures of sodium amidotrizoate and meglumine amidotrizoate (such as Gastrografin®, commercially available from Bristol-Myers Squibb, Princeton, N.J.), and, in general, the imaging contrast media mentioned previously in relation to X-rays.

PET scan imaging contrast media typically comprise a positron emitting (i.e. radioactive) element incorporated into a carrier such as a complexing agent or a biologically active molecule such as glucose. Specific PET scan imaging contrast media contemplated for use in the invention include, but are not limited to, complexes and derivatives of positron emitting radioisotopes including, but not limited to, carbon-11, nitrogen-13, oxygen-15, fluorine-18, iron-52, cobalt-55, copper-62, copper-64, bromine-75, bromine-76, technetium-94m, gallium-68, gallium 66, sellenium-73, bromine-75, bromine-76, iodine-120, iodine-124, and indium-110m. These radioactive elements may be incorporated into a carrier such as an organic molecule that is fluid at room temperature. Alternatively, these radioisotopes may be complexed with a complexing agent such as the complexing agents previously mentioned in regards to MRI imaging contrast media and placed in solution. Because the PET imaging contrast media are to be used in the expandable members placed inside the body, it may be preferable to choose PET imaging contrast media with short half-lives to reduce the risk to the patient in the event of a rupture of the expandable member. For example, PET imaging contrast media with a half-life of about 2 hours such as gallium-68 are preferred. PET scans can be adopted to the embodiments simply by injecting an applicable PET imaging contrast medium into the expandable member, thereby rendering the expandable member easily detectible by the PET scanning instrument.

In another embodiment of the invention, the imaging contrast media may include a metallic radioisotope including, but not limited to, the isotopes actinium-225, astatine-211, iodine-120, iodine-123, iodine-124, iodine-125, iodine-126, iodine-131, iodine-133, bismuth-212, arsenic-72, bromine-75, bromine-76, bromine-77, indium-110, indium-111, indium-113m, gallium-67, gallium-68, strontium-83, zirconium-89, ruthenium-95, ruthenium-97, ruthenium-103, ruthenium-105, mercury-107, mercury-203, rhenium-186, rhenium-188, tellurium-121m, tellurium-122m, tellurium-125m, thulium-165, thulium-167, thulium-168, technetium-94m, technetium-99m, fluorine-18, silver-11, platinum-197, palladium-109, copper-62, copper-64, copper-67, phosphorus-32, phosphorus-33, yttrium-86, yttrium-90, scandium-47, samarium-153, lutetium-177, rhodium-105, praseodymium-142, praseodyinium-143, terbium-161, holmium-166, gold-199, cobalt-57, cobalt-58, chromium-51, iron-59, selenium-75, thallium-201, and ytterbium-169.

FIG. 1, embodiments A and B, illustrate an exemplary device according to embodiments of the invention. A syringe 12 may be detachably connected to a longitudinal element 11 and 14, for example, using a luer lock 13 or other such device. An expandable member 10 may be connected to and in fluid communication with the distal end of the longitudinal element. In embodiment A, the longitudinal element 11 ends at the expandable member. In embodiment B, the longitudinal element 14 extends into the expandable member 10. The longitudinal element also could extend all the way to the distal end of the expandable member, or the expandable member could be positioned along the length of longitudinal element, as is known in the balloon catheter art.

FIG. 3 illustrates the exemplary inflation of an expandable member. FIG. 3 depicts a syringe 32 detachably connected using a luer lock 33 to a longitudinal element 31. An imaging contrast medium 35 has been drawn into the syringe 32 and is used to inflate the expandable member 30, which is shown in the expanded state.

In a preferred embodiment, a pressure measurement device may be connected to and in communication with the proximate end of the longitudinal element. The pressure measurement device may be used to monitor the pressure of the imaging contrast medium as it is delivered to the longitudinal element and the connected expandable member. The pressure measurement device may be, for example, a pressure transducer or pressure gauge. Preferably, a set point may be chosen so as to prevent rupture of the expandable member. Also, the set point preferably may be chosen to prevent unintended damage to adjacent tissues due to excessive forces imparted by the inflating expandable member. Furthermore, the set point preferably may be chosen to ensure that the expandable member has expanded to fill all of the unoccupied intervertebral disc space before inflation is stopped. One who is skilled in the art will be able to choose an appropriate set point at which inflation of the expandable member may be stopped, in accordance with the description herein.

FIG. 5 illustrates a preferred embodiment of the invention wherein a pressure measurement device is connected to and in communication with the proximate end of the longitudinal element. A syringe 52 may detachably connected to the proximate end of the longitudinal element 51, for example, using a luer lock 53 or other such device. A pressure measurement device 54, for example a pressure transducer or pressure gauge, also may be connected to the proximate end of the longitudinal element 51. An expandable member 50 may be connected to and in communication with the distal end of the longitudinal element.

In another preferred embodiment, a second catheter, cannula, or trocar may be coaxial to the longitudinal element. The second catheter, cannula, or trocar may function as a guide to facilitate insertion of the longitudinal element and the expandable member. The second catheter, cannula, or trocar preferably may sheath the longitudinal element and the expandable member that is connected to and in communication with the longitudinal element. Also, the distal end of the longitudinal element and the expandable member preferably may be extensible beyond the distal end of the second catheter, cannula, or trocar. In this way, the second catheter, cannula, or trocar may act as a sheath or sleeve to facilitate insertion of the longitudinal element and the expandable member into the body. When the second catheter, cannula, or trocar reaches or comes near to the intervertebral disc space, the longitudinal element may be extended beyond the distal end of the second catheter, cannula, or trocar in order to deliver the expandable member to the intervertebral disc space.

The extension of the expandable member and longitudinal element beyond the distal end of the second catheter, cannula, or trocar may be accomplished by preferential lengthening of the longitudinal element itself. For example, a flexible section of the longitudinal element may extend so as to lengthen the longitudinal element. Alternatively, the second catheter, cannula, or trocar may be retractable, so as to preferentially allow the longitudinal element and expandable member to extend beyond its distal end.

As with the longitudinal element, the second catheter, cannula, or trocar may have an optimally chosen flexibility and diameter. In a preferred embodiment, because the second catheter, cannula, or trocar may sheath the expandable member and the longitudinal element, the diameter of the second catheter, cannula, or trocar will be larger that the external diameter of the longitudinal element and large enough to enclose the expandable member in its deflated state. One of skill in the art will be capable of selecting an appropriate second catheter, cannula, or trocar, using the guidelines herein.

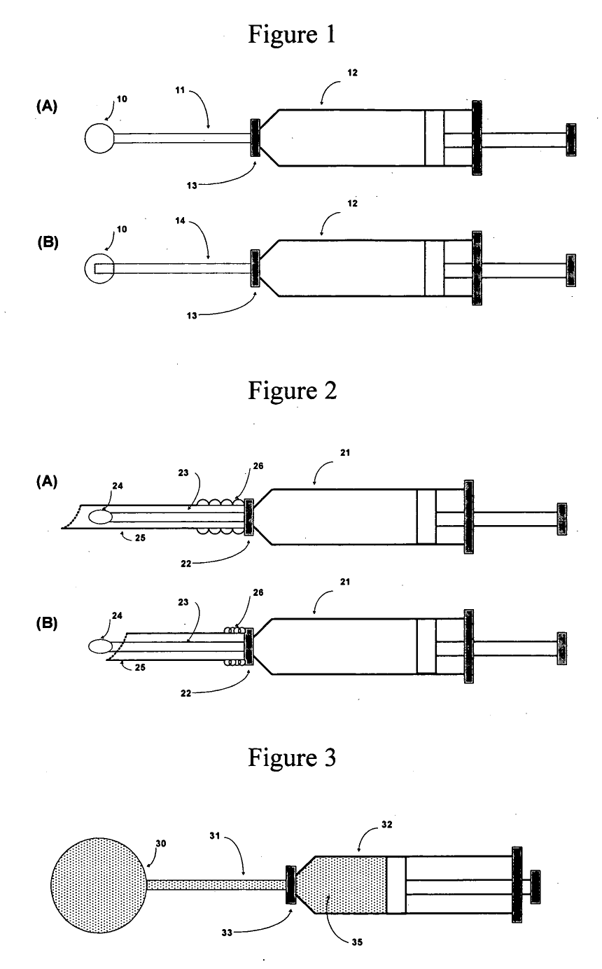

FIG. 2, embodiments A and B, illustrate an exemplary device according to a preferred embodiment of the invention. A syringe 21 may be detachably connected to a longitudinal element 23, for example, using a luer lock 22 or other such device. An expandable member 24 may be connected to and in communication with the distal end of the longitudinal element 23. A second catheter, cannula, or trocar 25 may sheath the expandable member 24 and longitudinal element 23. In embodiment A, the second catheter, cannula, or trocar 25 is illustrated in an extended position where the expandable member 24 and longitudinal element 23 are fully sheathed. In embodiment B, the second catheter, cannula, or trocar 25 is in a retracted position where the expandable member 24 and the distal end of the longitudinal element 23 are extended beyond the distal end of the second catheter, cannula, or trocar 25. In this way, the second catheter, cannula, or trocar 25 may aid in delivery of the expandable member 24 to the intervertebral disc space by sheathing the expandable member during insertion (embodiment A) until placed adjacent to the intervertebral disc space, at which time the expandable member 24 may be extended beyond the distal end of the second catheter, cannula, or trocar 25 for insertion into the intervertebral disc space. In this exemplary embodiment, a flexible or collapsible section 26 of second catheter, cannula, or trocar 25 enables it to be retracted.

In another preferred embodiment, the device may additionally comprise a guidewire positioned within the longitudinal element. The guidewire may be used to guide the longitudinal element during insertion so as to more easily place the longitudinal element at the desired position in the body, for example immediately adjacent to or inside of the intervertebral disc space.

Preferably, the longitudinal element may be able to pivot or flex in order to deform the longitudinal element from a linear to a bent or curved configuration. For example, if a guidewire is provided in the longitudinal element and is connected to the distal end of the longitudinal element, retracting the guidewire may cause the longitudinal element to bend or flex. In another embodiment, if a guide catheter or cannula is provided, the guide catheter or cannula may be bent or flexed in order to cause the longitudinal element disposed therein to bend or flex. A selectively flexible longitudinal element may be advantageous in order to facilitate insertion of the longitudinal element and the expandable member disposed thereon into the confines of the intervertebral disc space. For example, as the distal end of the longitudinal element and the expandable member are inserted into the disc space, it may be desirable to bend or flex the longitudinal member so that it better conforms to the disc space and can reach sufficiently far into the confines of the disc space in order to deliver the expandable member therein.

In another embodiment, there is provided a surgical kit. The surgical kit may comprise a longitudinal element having distal and proximate ends; an expandable member; a syringe; and an optional imaging contrast medium. In a preferred embodiment, the kit may further comprise a pressure measurement device, a second catheter, cannula, or trocar having distal and proximate ends, and a guidewire.

Each component of the kit may be connected or detached but connectible. For example, the expandable member may be connected to the distal end of the longitudinal element, or may be detached but connectible to the distal end of the longitudinal element. Likewise, the syringe and pressure measurement device each may be connected to the proximate end of the longitudinal element or may be detached but connectible to the proximate end of the longitudinal element. Similarly, the second catheter, cannula, or trocar may already sheath the longitudinal element and the expandable member or may be separate from, but capable of being slipped over, the longitudinal element and the expandable member.

Preferred embodiments also include methods of characterizing the intervertebral disc space using an expandable membrane. Specifically, preferred embodiments include methods for determining parameters such as the volume, dimensions, and geometry of an intervertebral disc space.

In an exemplary embodiment, and expandable member is inserted into the intervertebral disc space. Preferably, the expandable member may be inserted using minimally invasive surgical techniques. For example, a longitudinal element such as described herein may be used to insert the expandable member into the intervertebral disc space. One who is skilled in the art will appreciate other ways in which to introduce an expandable member into an intervertebral disc space.

The intervertebral disc space may be partially or fully evacuated before insertion of the expandable member. Partial or full evacuation of the intervertebral disc space may be accomplished, for example, by removing at least a portion of the nucleus and/or annulus of the intervertebral disc space before insertion of the expandable member. For example, a degenerated or undesired portion of the nucleus and/or annulus of the intervertebral disc may be removed before insertion of the expandable member. Alternatively, a complete nucleotomy or discectomy may be performed to remove the nucleus or entire intervertebral disc before insertion of the expandable member. Methods of accessing the disc space, and removing a portion or all of the nucleus and/or annulus are well known in the art, and applicable for use in the embodiments disclosed herein.

The expandable member, for example, may be a balloon. It is preferred that the expandable member be the balloon described herein. That is, preferably, the expandable member is a balloon made of various polymeric materials such as polyethylene terephthalates, polyolefins, polyurethanes, nylon, polyvinyl chloride, silicone, polyetherketone, polylactide, polyglycolide, poly(lactide-co-glycolide), poly(dioxanone), poly([epsilon]-caprolactone), poly(hydroxylbutyrate), poly(hydroxylvalerate), tyrosine-based polycarbonate, polypropylene fumarate, and combinations thereof.

The expandable member may be inflated with an imaging contrast medium. The imaging contrast medium may be any applicable imaging contrast agent, including, for example, X-ray, CT scan, MRI, and PET scan imaging contrast media. For example, the imaging contrast media described herein preferably may be delivered to the expandable member. In a preferred method of delivering the imaging contrast medium, a pressure measurement device may be used to measure the pressure of the imaging contrast medium during inflation of the expandable member. The expandable member may be considered inflated when the pressure of the imaging contrast medium has reached a pre-determined set point. Preferably, the set point may be chosen so as to prevent rupture of the expandable member. Also, the set point preferably may be chosen to prevent unintended damage to adjacent tissues due to forces imparted by the inflating expandable member. Furthermore, the set point preferably may be chosen to ensure that the expandable member has expanded to fill all of the unoccupied intervertebral disc space before inflation is stopped. The pressure gauge also could be used to determine when the expandable member is fully expanded by visually inspecting the gauge. In this embodiment, the expandable member has been filled when the pressure rises rapidly. One who is skilled in the art will be able to select an appropriate set point at which inflation of the expandable member may be stopped, in accordance with the description herein. It also may be preferred that inflation of the expandable member be accomplished in a minimally invasive fashion.

The volume of the inflated expandable member also preferably may be measured. In one embodiment, the volume of imaging contrast medium used to inflate the expandable member may be measured during inflation. For example, the volume displacement of a syringe by which imaging contrast medium is delivered to the expandable member in order to inflate the expandable member may be measured. It may be necessary to subtract from the measurement of the volume displacement of the syringe the volume of a catheter, cannula, trocar, or other mechanism by which the syringe is connected to the expandable member. In this way, the volume of the intervertebral disc space occupied by the expandable member may be determined. In another embodiment, the volume of imaging contrast medium extracted from the expandable member during deflation of the expandable member may be measured. Again, this may allow the volume of the intervertebral disc space occupied by the expandable member to be determined. By measuring the volume of imaging contrast agent used to inflate the expandable member, the occupied volume of the intervertebral disc space can be inferred. This may help in selecting an appropriate spinal implant to fit the intervertebral disc space. Additionally, direct measurement of the intervertebral disc volume may yield a more accurate determination of the volume of the intervertebral disc space than radiographic measurement alone would yield.

In another preferred embodiment, the inflated expandable member may be imaged in order to measure at least one parameter of the intervertebral disc space. Parameters that can be measured according to embodiments of the invention include one-dimensional parameters such as the anterior-posterior width, lateral width, and height of the intervertebral disc space. One-dimensional parameters preferably are measured by X-ray (e.g. fluoroscopy). Additionally, two-dimensional parameters such as the cross-sectional areas of the intervertebral disc space perpendicular (i.e. “footprint”) and parallel (i.e. “projected”) to the spinal column can be determined. Simple imaging techniques such as X-ray may be useful to determine the cross-sectional area of the intervertebral disc space parallel to the spinal column, but more advanced imaging techniques such as CT, C-arm fluoroscopy, MRI, and PET technologies preferably are used to determine the cross-sectional area of the disc space perpendicular to the spinal column. Additionally, three-dimensional parameters of the intervertebral disc space such as the volume and geometry (e.g. topography) of the disc space may be determined.

Where a computerized imaging technique is used, parameters of the disc space may be determined by a computer analyzing the obtained images. For example, a computer may directly compute the volume of the intervertebral disc space or cross-sectional areas of the disc space. In both computational and non-computational imaging techniques, it may be advantageous to include a dimensional reference in the images in order to normalize the observed dimensions of the disc space. For example, a metal structure such as a rod of known dimensions may be placed adjacent to the intervertebral disc space (e.g. on the skin of the patient at a location adjacent to the disc space) prior to imaging such that the rod will appear in the images obtained of the disk space. In this manner, the length of dimensions observed in the images may be normalized to the known length of the dimensional reference.

One who is skilled in the art will appreciate the existing procedures and methods by which intra-operative radiography may be carried out. The measurements obtained may be used to size a spinal implant prior to implantation. Sizing prior to implantation may be advantageous because of the reduced surgical time and increased likelihood of a desirable clinical result. Measurements of the intervertebral disc space's parameters may be made, for example, by manually examining the images created by imaging the inflated expandable member or by computer computation of the dimensions and geometry based on the images obtained. Use of this method provides improved determination of disc space parameters, when compared to use of inflatable expandable members filled with air or other non-radiographic or non-imaging medium, like water or saline.

Various parameters of the intervertebral disc space may be determined by imaging the expandable member inflated with the imaging contrast medium. Preferably, the height of the intervertebral disc space and footprint area are measured by imaging of the inflated expandable member. Measuring the height of the intervertebral disc space may include measuring the anterior, middle, and posterior height of the disc space, as these three measurements may differ, even in the same intervertebral disc space. Additionally, the wedge angle of the intervertebral disc space may be measured.

In a more preferred embodiment, the geometry of the disc space also is examined by imaging of the inflated expandable member. Generally, the vertebral end plates of an intervertebral disc may be concave, convex, flat, or irregularly shaped. Additionally, the cross-sectional shape of the intervertebral disc space (usually described as kidney-like) may vary slightly from patient to patient. Determination of the geometry of the disc space therefore may enable further customization and sizing of the spinal implant to the individual patient. Additional parameters of the intervertebral disc space that may be measured include the location of the endplate/nucleus boundary and the location of the annulus/nucleus boundary.

The inflated expandable member may be imaged with any applicable imaging regime, technique, or technology. Preferred methods of imaging the inflated expandable member include X-ray, derivative X-ray technologies such as CT (computerized tomography) and C-arm fluoroscopy (e.g. Iso-C technology available from Siemens AG, Berlin, Germany), MRI, and PET scan. In a preferred embodiment, the imaging contrast medium may be selected to correspond to the method of imaging that is to be used. The inflated expandable member may be imaged once or a multiple of times. In another embodiment of the invention, more than one imaging method may be used. If more than one imaging method is to be used, it may be preferable to inflate the expandable member with an imaging contrast medium appropriate for one of the imaging methods, deflate the expandable member, and then inflate the expandable member again, but with a different imaging contrast medium appropriate for another imaging method. This may be repeated for each imaging method to be used.

The expandable member may be deflated following inflation with the imaging contrast medium. Deflation of the expandable member may facilitate removal of the expandable member from the intervertebral disc space. In a preferred embodiment, the expandable member is deflated using minimally invasive surgical techniques. Therefore, the imaging contrast medium preferably may be removed from the expandable member in a method analogous to the manner in which the imaging contrast medium was first delivered to the expandable member. For example, if the imaging contrast medium were used to inflate the expandable member by delivering the medium through a cannula into the expandable member, then the imaging contrast medium preferably may be removed from the expandable member through the same cannula sued to inject the medium. In another embodiment, the imaging contrast medium may be removed from the expandable member by puncturing the expandable member with, for example, a hypodermic needle or a trocar and withdrawing the imaging contrast medium through the needle or trocar.

Following deflation, the expandable member may be removed from the intervertebral disc space. Preferably, the expandable member may be removed from the intervertebral disc space in a minimally invasive manner. For example, the expandable member may be removed from the intervertebral disc space using a catheter, cannula, or trocar through which the expandable member is extracted. In a more preferred embodiment, the same catheter, cannula, or trocar that was used to insert the expandable member into the intervertebral disc space also may be used to remove the expandable member from the disc space. In this way, unnecessary damage to surrounding tissues preferably may be avoided.

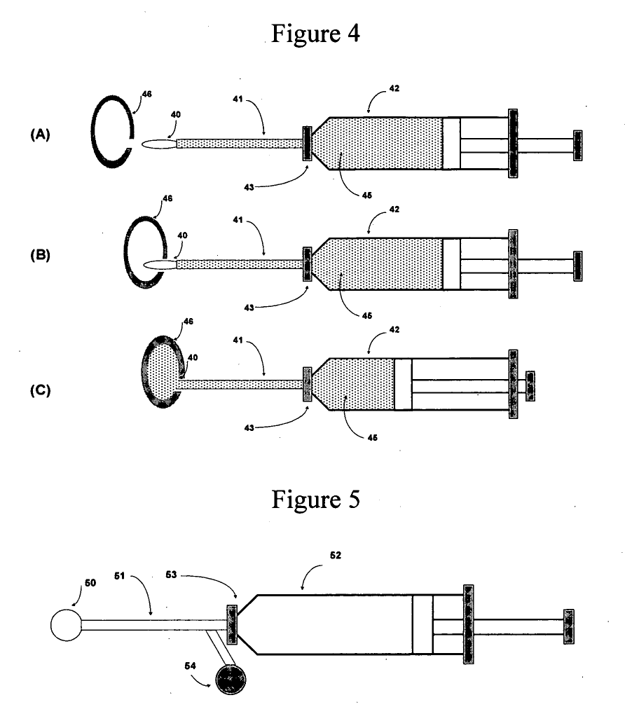

FIG. 4, embodiments A, B, and C, illustrate an exemplary embodiment where a syringe 42 may be detachably connected using a luer lock 43 to a longitudinal element 41. An expandable member 40 may be connected to and in fluid communication with the longitudinal element 41. An imaging contrast medium 45 may be drawn into the syringe 45 and used to inflate the expandable member 40. In embodiment A, the device is shown approaching an intervertebral disc. In embodiment B, the expandable member 40 is inserted into the intervertebral disc space. In embodiment C, the expandable member 40 is inflated with the imaging contrast medium 45 to fill the intervertebral disc space.

The systems and methods of the invention may be advantageously used to determine various intervertebral disc parameters such as the volume, dimensions, and geometry prior to implantation of a spinal implant. The spinal implant may be any implant used to replace all or part of the nucleus and/or annulus of the intervertebral disc, for example a fusion cage, artificial disc, or prosthetic disc nucleus. A snug fit between the spinal implant and the intervertebral disc space is thought to be desirable because of the reduced possibility of implant rotation, reduced possibility of excessive implant movement inside the disc space, increased contact between the vertebral end plates and implant, and increased annulus tension. Therefore, a correctly sized spinal implant may be more likely to achieve a desirable clinical result than would be an incorrectly sized implant.

For example, it may be preferred that an spinal implant be no more than about 125%, more preferably no more than about 120%, and most preferably no more than about 115% of the volume of the intervertebral disc space in which it is to be implanted. It is thought that the spinal implant can displace some amount of tissue upon implantation in the intervertebral disc space, thereby allowing a small amount of oversizing. However, it is thought that a spinal implant greater than about 125% of the volume of the intervertebral disc space in which it is to be implanted will be too difficult to implant because of its excessive size. It also may be preferable to avoid substantial undersizing of the spinal implant. For example, an implant no more that about 10% to about 15% undersized may be preferable. The invention may enable an intervertebral disc implant to be sized within about 20-25%, more preferably within about 15%, and most preferably within about 10% of the volume of the intervertebral disc space in which it is to be implanted.

In another embodiment of the invention, excess tissue may be removed before implantation of the spinal implant. Because implants typically are manufactured pre-surgery, it may be easier to shape the intervertebral disc space to fit the implant than it is to shape the implant to conform to the intervertebral disc space. Imaging of the inflated expandable member and determination of various parameters of the disc space such as the dimensions, volume, and geometry of the intervertebral disc space therefore may enable a surgeon to determine what, if any, excess tissue should be removed prior to implantation of the spinal implant. This may lead to a closer correlation in size and shape between the intervertebral disc space and the spinal implant, and a more desirable clinical outcome.

The invention now will be described in more detail with reference to the following non-limiting examples.

### EXAMPLES

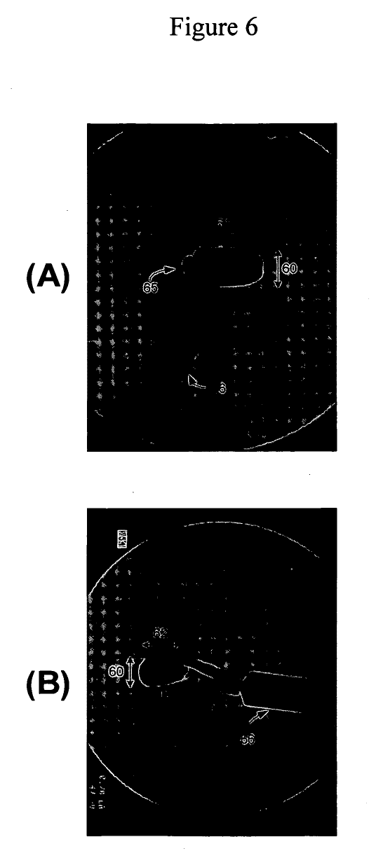

FIG. 6, embodiments A and B, illustrates the in-vivo use of an exemplary device according to embodiments of the invention. A latex expandable member 65 was attached to a syringe 66 using surgical tubing. The expandable member 65 was inserted into the intervertebral disc space. An imaging contrast medium was used to inflate the expandable member and X-rays were taken of the inflated member. 60 indicates the height of the intervertebral disc space as seen in both embodiments. In embodiment A, the lateral width 61 of the intervertebral disc space is visible. In embodiment B, the posterior-anterior width 62 of the intervertebral disc space is visible. Determination of these dimensions, along with the volume of contrast medium necessary to inflate the expandable member, may enable an intervertebral disc prosthesis to be sized according to the disk space, thereby increasing the likelihood of an acceptable clinical outcome following implantation of the prosthesis. As can be seen, use of the imaging contrast medium significantly helps to distinguish the adjacent vertebrae from the disc space in which the expandable member has been inflated.

What is claimed is:

1. A method for determining at least one parameter of an intervertebral disc space, comprising:
inserting an expandable member into an at least partially evacuated intervertebral disc space;
inflating the expandable member with an imaging contrast medium;
imaging at least the intervertebral disc space when the expandable member has sufficiently occupied the at least partially evacuated disc space; and
measuring the volume of imaging contrast medium in the expandable member thereby determining at least one parameter of the intervertebral disc space.

2. The method of claim 1, wherein measuring the volume of imaging contrast medium comprises measuring the volume of imaging contrast medium as it is delivered to the expandable member.

3. The method of claim 1, further comprising deflating the expandable member, and where measuring the volume of imaging contrast medium comprises measuring the volume of imaging contrast medium removed from the expandable member during deflation of the expandable member.

4. The method of claim 1, wherein imaging the expandable member comprises an imaging procedure selected from the group consisting of X-ray, C-arm fluoroscopy, CT scan, MRI, and PET scans.

5. The method of claim 1, wherein the parameter is a dimension selected from the group consisting of the height, posterior-anterior width, and lateral width of the intervertebral disc space.

6. The method of claim 1, wherein the parameter is a two-dimensional parameter selected from the footprint area and projected area of the intervertebral disc space.

7. The method of claim 1, wherein the parameter is a three-dimensional parameter selected from the geometry and volume of the intervertebral disc space.

8. The method of claim 1, further comprising determining a characteristic of the intervertebral disc space selected from the group consisting of the concave or convex nature of the vertebral end plates, the location of the endplate/nucleus boundary, the location of the annulus/nucleus boundary, and the shape of the intervertebral disc space.

9. The method of claim 1, wherein inserting an expandable member and inflating the expandable member are carried out using minimally invasive surgical techniques.

10. The method of claim 1, wherein the expandable member is a balloon.

11. The method of claim 1, wherein the imaging contrast medium is selected from the group consisting of X-ray, C-arm fluoroscopy, CT scan, MRI, and PET scan imaging contrast media.

12. The method of claim 1, further comprising monitoring the pressure of the imaging contrast medium during inflation of the expandable member and stopping inflation when the pressure reaches a predetermined set point.

13. A method for determining at least one parameter of an intervertebral disc space, comprising:
removing at least a portion of a nucleus of the intervertebral disc;
inserting an expandable member into the disc space;
inflating the expandable member with an imaging contrast medium;
measuring the volume of imaging contrast medium used to inflate the expandable member;
imaging the intervertebral disc space while the expandable member is inflated with the imaging contrast medium to determine when the expandable member has sufficiently occupied the disc space; and
calculating the volume of imaging contrast medium in the expandable member to determine the at least one parameter.

14. The method of claim 13, wherein removing at least a portion of the nucleus of the intervertebral disc space, inserting an expandable member, and inflating the expandable member are carried out using minimally invasive surgical techniques.

15. The method of claim 13, wherein the expandable member is a balloon.

16. The method of claim 13, further comprising monitoring the pressure of the imaging contrast medium during inflation and stopping inflation when the pressure reaches a predetermined set point.

17. The method of claim 13, wherein the imaging contrast medium is selected from the group consisting of X-ray, C-arm fluoroscopy, CT scan, MRI, and PET scan imaging contrast media.

18. The method of claim 13, wherein measuring the volume of imaging contrast medium comprises measuring the volume of imaging contrast medium as it is delivered to the expandable member.

19. The method of claim 13, further comprising deflating the expandable member, and wherein measuring the volume of imaging contrast medium comprises measuring the volume of imaging contrast medium removed from the expandable member during deflation of the expandable member.

20. The method of claim 13, wherein imaging the expandable member comprises an imaging procedure selected from the group consisting of an X-ray, C-arm fluoroscopy, CT scan, MRI, and PET scan.

21. The method of claim 13, wherein the parameter is a dimension selected from the group consisting of the height, posterior-anterior width, and lateral width of the intervertebral disc space.

22. The method of claim 13, wherein the parameter is a two-dimensional parameter selected from the footprint area and projected area of the intervertebral disc space.

23. The method of claim 13, wherein the parameter is a three-dimensional parameter selected from the geometry and volume of the intervertebral disc space.

24. The method of claim 13, further comprising determining a characteristic of the intervertebral disc space selected from the group consisting of the concave or convex nature of the vertebral end plates, the location of the endplate/nucleus boundary, the location of the annulus/nucleus boundary, and the shape of the intervertebral disc space.

25. An intradisc sizer, comprising:
a longitudinal element comprising distal and proximate ends;
an expandable member comprising an internal cavity connected to and in fluid communication with the distal end of the longitudinal element;
a dispensing device adapted to be connected to the proximate end of the longitudinal element; and
an imaging contrast medium positionable within the dispensing device.

26. The device of claim 25, wherein the imaging contrast medium is selected from the group consisting of X-ray, C-arm fluoroscopy, CT scan, MRI, and PET scan imaging contrast media.

27. The device of claim 25, wherein the expandable member is an unconstrained balloon.

28. The device of claim 25, wherein the expandable member comprises a polymeric material selected from the group consisting of polyethylene terephthalates, polyolefins, polyurethanes, nylon, polyvinyl chloride, silicone, polyetherketone, polylactide, polyglycolide, poly(lactide-co-glycolide), poly(dioxanone), poly([epsilon]-caprolactone), poly(hydroxylbutyrate), poly(hydroxylvaierate), tyrosine-based polycarbonate, polypropylene fumarate, and mixtures and combinations thereof.

29. The device of claim 25, wherein the dispensing device is a syringe graduated by volume.

30. The device of claim 25, further comprising a pressure measurement device connected to and in communication with the proximate end of the longitudinal element.

31. The device of claim 25, further comprising a guidewire positioned within the longitudinal element.

32. The device of claim 25, further comprising a guide cannula or catheter, wherein the longitudinal element and expandable member is capable of being positioned within the guide cannula or catheter.

33. The device of claim 25, wherein the longitudinal element is capable of being selectively pivoted between a linear and curved configuration.

34. A kit comprising:
a longitudinal element comprising distal and proximate ends;
an expandable member connected to and in fluid communication with the distal end of the longitudinal element;
a dispensing device adapted to be connected to the proximate end of the longitudinal element; and
an imaging contrast medium.

35. The kit of claim 34, wherein the imaging contrast medium is selected from the group consisting of X-ray, C-arm fluoroscopy, CT scan, MRI, and PET scan imaging contrast media.

36. The kit of claim 34, wherein the expandable member is an unconstrained balloon.

37. The kit of claim 34, wherein the expandable member comprises a polymeric material selected from the group consisting of polyethylene terephthalates, polyolefins, polyurethanes, nylon, polyvinyl chloride, silicone, polyetherketone, polylactide, polyglycolide, poly(lactide-co-glycolide), poly(dioxanone), poly([epsilon]-caprolactone), poly(hydroxylbutyrate), poly(hydroxylvalerate), tyrosine-based polycarbonate, polypropylene fumarate, and mixtures and combinations thereof.

38. The kit of claim 34, wherein the dispensing device is a syringe graduated by volume.

39. The kit of claim 34, further comprising a pressure measurement device that is detachably connectible to the proximate end of the longitudinal element.

40. The kit of claim 34, further comprising a guidewire capable of being positioned within the longitudinal element.

41. The kit of claim 34, further comprising a guide cannula or catheter, wherein the longitudinal element and expandable member is capable of being positioned within the guide cannula or catheter.
